(19) United States
(12) Patent Application Publication
(10) Pub. No.: US 2016/0130641 A1
(43) Pub. Date: May 12, 2016

(54) HIGHLY SENSITIVE METHOD FOR DETECTING LOW FREQUENCY MUTATIONS

(71) Applicant: Janssen Diagnostics, LLC, Raritan, NJ (US)

(72) Inventors: Haiying Wang, Bridgewater, NJ (US); Yuqiu Jiang, Flemington, NJ (US); Yixin Wang, Basking Ridge, NJ (US)

(21) Appl. No.: 14/375,894

(22) PCT Filed: Feb. 13, 2013
(86) PCT No.: PCT/US2013/025913

§ 371 Date: Jul. 31, 2014

Related U.S. Application Data
(60) Provisional application No. 61/599,074, filed on Feb. 15, 2012.

(51) Int. Cl. C12Q 1/68

(52) U.S. Cl. CPC C12Q 1/6827

(57) ABSTRACT

The disclosed edge-blocker oligonucleotide based AS-NEPB-PCR method amplifies allele specific DNA (or RNA) while dramatically blocking amplification of wild type (WT) DNA (or RNA). The AS-NEPB-PCR design allows ready modification of an existing PCR reaction setup with an edge-blocker oligonucleotide together with an allele specific primer complementary to the mutant sequence to achieve allele specific amplification. The method simplifies assay optimization procedures and achieved sensitivity sufficient to detect a signal present at 0.1% level with close to 100% specificity, which is useful in detecting SNP or genetic mutations. The method was used to detect three different genetic mutations in cancer, in KRAS, BRAF, and EGFR, with three different types of modified edge-blocker oligonucleotides (phosphate, inverted dT and amino-C7). It was possible to detect one copy of mutant DNA in 1000-copy of normal DNA background of a heterogeneous sample, and was far more sensitive than the other blocking method.

### BACKGROUND

Use of molecular assays is increasing rapidly in the diagnostic field. Molecular testing has been utilized by clinical scientists and physicians for better understanding of patient disease profiles as well as resistance to diseases at the molecular level. In particular, genetic differences between individuals are considered to be significant factors in evaluating response to treatment or disease and determine response to therapy.

The holy-grail in treating many diseases is to target particular aberrant and harmful cell types. Often such aberrant cells display mutations not present in the surrounding normal tissue. This is particularly true in the case of cancer where actual clones of cancerous cells may be far fewer in number than the normal cells in the surrounding normal tissue. Sometimes, and with increasing success, such aberrant cells can be targeted by therapies before they expand—provided their presence is detectable. Thus, in combination with clinical parameters molecular profiles can be used to assist in selecting a treatment for a patient, predicting the response of a patient to a particular therapy or predict the disease-free and overall survival for a patient.

In evaluating disease conditions like cancer, a patient may harbor cells that have one or more single point substitution, insertion, or deletion mutations that play a deleterious role. Different patients may have different mutations even when having the same ‘type’ of cancer. Each mutation or group of mutations may be best treated by different therapeutic regimes. But, such point mutations are often far more difficult to detect than large insertions or deletions or germ line mutations since they are camouflaged by normal tissue which yields a large signal due to the identity of virtually all of nucleic acid sequence flanking the mutation site with genetic material obtained from surrounding normal tissue in a sample. In other words, a sample of tissue including some cancerous tissue but also much normal tissue will, upon using Polymerase Chain Reaction (“PCR”) techniques, generally yield a signal corresponding to normal tissue rather than the mutant sequence.

Although with a sensitive technique like PCR, the usual admonition is to adopt special laboratory practices to avoid false positive amplifications because the high throughput and repetition of PCR assays can lead to amplification of a single DNA molecule. When the signal is buried in a sea of almost identical nucleic acid molecules additional technical problems need to be addressed to avoid amplifying the background of almost identical nucleic acid molecules instead of the desired target nucleic acid molecules because the background signal is due to molecules that have the advantage of numbers. As a result, a PCR based amplification may simply not readily amplify the nucleic acid variant present at a low frequency—typically present in a tumor cell since the variant is often present at a much lower level than normal tissue in a sample (often at just a fraction of a percent)—when attempting early detection of a cancer or detection and monitoring by relatively non-invasive means.

Utility of such an assay often depends to a great degree on successfully segregating cancerous tissue from normal tissue to enrich for the target nucleic acid variant before conducting a genotype analysis or on adopting procedures that greatly favor amplification of the target nucleic acid molecule instead of an almost identical sea of background molecules. Examples of such techniques are described by, for instance, Newton et al. in “Analysis of any point mutation in DNA: The amplification refractory mutation system (ARMS)” Nucleic Acids Res. 17:2503-2516 (1989). Sensitivity of such a technique may be expressed in terms of its sensitivity. A sensitivity of 1% indicates an ability to amplify and generate a signal corresponding to a target mutant molecular species present at a level of 1:100 in a sea of almost identical background normal copies.

A promising technique useful in the context of cancer is Allele-Specific amplification using Real-Time Polymerase Chain Reaction technology (“AS-RT-PCR”) that suppresses the amplification of the wild type sequence using a blocker oligonucleotide that preferentially binds to the wild type sequence to suppress its amplification relative to a variant of the wild type sequence. This technique helps evaluate genetic mutations present at a low frequency in a patient—such as in tumor cells or in a chimera. This technique can help detect genetic variants, single nucleotide polymorphisms (SNP) and genetic mutations present at low frequency—as is demonstrated herein later.

Example of AS-RT-PCR protocols and their limitations are described in, for instance, Morlan et al. “Mutation Detection by Real-Time PCR: A Simple, Robust and Highly Selective Method”, PLoS ONE 4(2): e4584. doi:10.1371/journal.pone.0004584(2009). The Morlan reference describes the use of center-blocker oligonucletides to enhance the detection of point mutations—which is an improvement of the allele specific amplification technique reported by Wu et al. in “Allele-specific enzymatic amplification of f8-globin genomic DNA for diagnosis of sickle cell anemia”, Proc. Natl. Acad. Sci. USA 86:2757-60 (1989). In the Morlan technique a mutant specific primer is used together with a center-blocker oligonucleotide. By center-blocker oligonucleotide is meant an oligonucleotide with a mismatch that is about equidistant from either end. The mutant specific primer is entirely complementary to the mutant sequence. The mutant specific primer of Morlan is further trimmed at its 5′ end to reduce its melting temperature to about 10° C. below the anneal/extend temperature used in the PCR. The center blocker oligonucleotide is complementary to the wild type sequence and spans the site of a point mutation so that the point mutation is about equally spanned by it. The center blocker oligonucleotide is further phosphorylated at its 3′ end to prevent extension during a PCR reaction.

Other example efforts include Dames et al. “Characterization of Aberrant Melting Peaks in Unlabeled Probe Assays”, Journal of Molecular Diagnostics, Vol. 9, No. 3, July 2007; and Willem Maat and Pieter A. Van der Velden, “Pyrophosphorolysis Detects B-RAF Mutations in Primary Uveal Melanoma”, Investigative Ophthalmology & Visual Science, January 2008, Vol. 49, No. 1.

With the center-blocker oligonucleotide technique, despite good results with detection of selected point mutations, and significant improvements over prior efforts, it remains a challenge to routinely achieve sensitivities of better than 1% to really utilize the technique to the fullest extent to detect rare mutations. Further, the technique is limited to the detection of point mutations. Sensitive detection of rare alleles that are not solely defined by point mutations continues to be an additional challenge.

### SUMMARY

Disclosed is a novel edge-blocker oligonucleotide based Non-Extendable Primer Blocker Allele Specific-Real Time Polymerase Chain Reaction (“AS-NEPB-PCR”) based mutation assay methodology that overcomes the limitations of prior art approaches to enable amplification and detection of nucleic acid variants present at a frequency lower than 1% to achieve selectivity for targets present at levels of less than 1%. The disclosed method enables a universal design of Allele Specific primer and primer blocker that can be used in any of AS-RT-PCR assays to detect SNP or genetic mutations. The method simplifies assay optimization procedures and achieved 0.1% detection sensitivity with close to 100% specificity.

The disclosed edge-blocker oligonucleotide based AS-NEPB-PCR method amplifies allele specific DNA (or RNA) while dramatically blocking amplification of wild type (WT) DNA (or RNA). The disclosed AS-NEPB-PCR design allows ready modification of an existing PCR reaction setup by introducing an edge-blocker oligonucleotide together with an allele specific primer complementary to the mutant sequence to achieve allele specific amplification. In a preferred embodiment the edge-blocker oligonucleotide and allele specific primer may have the same length and differ only at the 3′ end where the edge-blocker oligonucleotide has a non-complementary base relative to the mutant sequence and a blocked 3′ end while the allele specific primer is preferably entirely complementary to the mutant sequence of interest and has a hydroxyl group at its 3′ end to allow extension during a PCR reaction. This method is not only simpler to implement but also successful in routinely suppressing the amplification of the wild type sequence to almost undetectable levels even when the mutant sequence is present at a frequency of about 0.1% of the wild type sequence. Further, the edge-blocker oligonucleotide based AS-NEPB-PCR method is not limited to just detecting point mutations, but can detect specified insertions or deletions as well.

The edge-blocker oligonucleotide based AS-NEPB-PCR method was used to detect three different genetic mutations in cancers. The genetic mutations targeted here were in KRAS, BRAF, and EGFR genes, which were detected with the use of three different types of modified edge-blocker oligonucleotides (phosphate, inverted dT and amino-C7). The resulting data were compared to one of the known common blocking methods as a reference. The novel method disclosed herein was able to detect one copy of mutant DNA in 1000-copy of normal DNA background of a heterogeneous sample, and was far more sensitive than the reference blocking method.

A preferred method for detecting a mutant nucleic acid sequence, defined by one or more mutations due to at least one or more of a substitution, a deletion or an insertion, while suppressing the signal due to the wild type sequence includes several steps. Preferably a primer complementary to the mutant nucleic acid sequence is selected such that its 3′ end matches up with at least one mutated nucleic acid position. A second primer, an edge-blocker wild type oligonucleotide, is also used. The edge-blocker wild type oligonucleotide corresponds to the wild type sequence such that the 3′ end of the edge-blocker wild type primer has at least one mismatch at or about its 3′ end relative to the mutant nucleic acid sequence but has no mismatches relative to the wild type sequence. Further, the 3′ hydroxyl group at end of the edge-blocker wild type primer is blocked whereby making it non-extendable in a polymerase chain reaction. In a preferred embodiment reverse primers are selected as usual although it should be noted that when trying to detect deletions or insertions, it may be advantageous to use reverse primers similar to the one corresponding to the wild type sequence having a blocked 3′ end. The amplification products of a PCR reaction are detected with at least one probe specific for the amplified product in a polymerase chain reaction. Preferably, the polymerase chain reaction is a real-time polymerase chain reaction.

In a variation, one or more probes may be added after the polymerase chain reaction is initiated. Further, the initial starting materials may be generated using a reverse transcriptase to investigate transcription products for point or other mutations of interest.

An interesting situation handled by this method with ease is when two point mutations of interest are close to each other including being adjacent. In such a situation this method can use the 3′ end of the edge-blocker wild type oligonucleotide with at least one mismatch at or about its 3′ end relative to the mutant nucleic acid sequence—even two mismatches to cover both the point mutations to even more effectively suppress the amplification of the wild type sequence. It is preferred that the allele specific primer cover both the mutant positions. With such coverage even if there is extension based on the binding of the allele specific primer, the amplification products will correspond to the target mutations rather than the wild type sequence.

In an embodiment, as many as two out of the three base pairs immediately adjacent to the blocked 3′ end of the edge-blocker wild type oligonucleotide may have a mismatch, but may be counterbalanced by increasing the length of the edge-blocker wild type oligonucleotide to suppress amplification of the wild type sequence by the allele specific primer.

In a preferred embodiment, the edge-blocker wild type oligonucleotide is equal in length to the allele specific primer with both having 3′ ends that cover similar portions of the mutant or wild type sequence. The method can detect a mutant sequence even when it is present at a level of only about 1 in 1000 or even rarer.

However, as noted the edge-blocker wild type oligonucleotide may be longer at its 5′ end than the allele specific primer to assist it in competing out the allele specific primer to prevent accidental extension of the wild-type sequence by the allele specific primer.

To ensure effective competition by the edge-blocker wild type oligonucleotide the melting temperature of the allele specific primer is lower than that for the edge-blocker wild type oligonucleotide relative to the wild type sequence. Preferably, the melting temperature of the allele specific primer is lower, e.g., about 10° C. lower, than that for the edge-blocker wild type oligonucleotide.

Effective competition by the edge-blocker wild type oligonucleotide for the wild type sequence is helped by ensuring that the edge-blocker wild type oligonucleotide is present at a concentration suitable for suppression of the wild-type sequence while allowing amplification of the mutant sequence. Preferably, this concentration is comparable—while being at least equal—to the level of the wild type sequence concentration. The concentration of the allele specific primer, on the other hand, is in excess of that of the wild type sequence since it is incorporated into the PCR product while the edge-blocker wild type oligonucleotide serves to suppress amplification of the wild-type sequence, the likelihood of which decreases as the allele specific primer levels decrease with amplification of the target PCR product. Thus, preferably, the concentrations of the edge-blocker wild type oligonucleotide and the allele specific primer are comparable.

Preferably, using, for instance calibration curves, the disclosed method for the detection of rare mutant nucleic acid sequence includes quantitation to estimate a level of the mutant nucleic acid sequence relative to the wild type sequence. Such a calibration curve may be generated by spiking the samples for a polymerase chain reaction.

Early detection of cancer to better provide therapeutic intervention is made possible when the disclosed method is used to detect tumor cells by the mutant nucleic acid sequence corresponding to a tumor cell type in a tissue, CTCs or other sample collected from a patient. If the rare target allele corresponds to metastatic cell disease, intervention before the tumor cells become noticeable becomes a more realistic possibility.

Thus, the disclosed method is a diagnostic method suitable for early detection of cancer by way of detecting the presence of one or more target cells in a sample derived from a patient, which cells harbor a mutant nucleic acid sequence, and the presence of which cells likely leads to malignancy or recurrence. The method comprises selecting an allele specific primer corresponding to a portion of the mutant nucleic acid sequence such that the 3′ end of the allele specific primer does not have a mismatch while being aligned with at least one mutated nucleic acid position in a target. Also used is an edge-blocker wild type oligonucleotide corresponding to the wild type sequence such that the 3′ end of the edge-blocker wild type oligonucleotide has at least one mismatch at or about its 3′ end, and, wherein, furthermore, the 3′ end of the edge-blocker wild type oligonucleotide is blocked whereby making it non-extendable competitive inhibitor in a polymerase chain reaction. Together with other ingredients and one or more probes to detect the desired amplification products, a polymerase chain reaction, preferably a real time polymerase chain reaction is carried out. The method not only detects cancer usefully early, but also can guide one to therapies best suited for treating the patient.

As a check on spurious signals, the mutant nucleic acid sequence is detected by detecting the reaction products less than a pre-specified number of amplification cycles.

As a further safeguard against spurious signals, mutant nucleic acid sequence's presence is detected if amplification products corresponding to it are detected but a reference sequence, treated like the mutant sequence is not detected in the same sample. The target sequence, with or without its corresponding wild-type like sequence, may be used to spike the sample. This can determine sensitivity and other parameters of interest.

The edge-blocker wild type oligonucleotide is blocked by derivatizing or replacing its 3′ hydroxyl group with one or more selected from the group consisting of phosphate, inverted dT and amino-C7.

These and other benefits of the disclosed novel AS-NEPB-PCR method and variations thereof are described next with the aid of the included Figures.

### BRIEF DESCRIPTION OF THE FIGURES

FIG. 1. General diagram for edge-blocker oligonucleotide based AS-NEPB-PCR design: Primer with 3′ end modification (phosphate or inverted dT) functions as a blocking group to prevent polymerase extension on wild type sequence.—PCR amplifies only AS primed mutation while WT strain is blocked by a modified non-extendable primer (edge-blocker oligonucleotide based AS-NEPB-PCR).

FIG. 2. Mapping of edge-blocker oligonucleotide designs for V600E. AS Primer-1 and 2 indicate BRAF-AS-Forward Primer-1 and 2 (AS Primer-1 is 6 bases longer than AS Primer-2 at 5′) corresponding to Seq. Id. 1 and Seq. Id. 2 respectively. Edge-blocker oligonucleotide based AS-NEPB-PCR: WT-1 and 2 stand for edge-blocker oligonucleotide-1 and edge-blocker oligonucleotide-2 and they correspond to Seq Id. 5 and Seq. Id. 6 respectively. Probe-dye (Dye labeled probe—Seq. Id. 4) and reverse primer (Seq. Id. 3) are common for both assays. AS primer-1, Seq. Id. 1, runs with edge-blocker oligonucleotide-WT-1 (NEPB-WT-1), Seq. Id. 5, and AS primer-2, Seq. Id. 2, with edge-blocker oligonucleotide-WT-2 (NEPB-WT-2), Seq. Id. 6. Bold letters in legends and in the figures represent positions for mismatch, such as ‘A-BRAF mutation (V600E; T>A)’ in AS Primer 1 and 2; ‘*’ represents 3′ modifications here and in the figures.

FIG. 3. Mapping of edge-blocker oligonucleotide designs for two K-ras gene mutations. AS Primer-KrasP4 (Seq. Id. 17) and KrasP7 (Seq. Id. 21) indicate G12V-AS-Forward Primer and G13D-AS-Forward Primer. NEPB-WT-KrasP4B (Seq. Id. 18) and P7B (Seq. Id. 22) stand for G12V-NE edge-blocker oligonucleotide and G13D-NE edge-blocker oligonucleotide. KProbe1 (Seq. Id. 20) and KProbe2 (Seq. Id. 24) are Dye labeled probes, and reverse primer is common for both assays. AS Primer-KrasP4 (Seq. Id. 17) runs with edge-blocker oligonucleotide-WT-KrasP4B (Seq. Id. 18) and KProbe1 (Seq. Id. 20). AS Primer-KrasP7 (Seq. Id. 21) runs with center-blocker oligonucleotide-WT-KrasP7B (Seq. Id. 22) and KProbe2 (Seq. Id. 24). Red “t” indicated K-ras p.G12V; mutation (c.G>T) and Red “a” indicated K-ras p.G13D; mutation (c.G>A) in AS Primer-KrasP4 and P7 and as usual ‘*’ represents -3′ modifications.

FIG. 4. BRAF V600E mutant detection: 8 out of 20 ul PCR products from the 20 ng DNA reaction were loaded on the gel. The single sharp bands were observed from 5% to 0.1% mutant reactions and no PCR products were observed from SKBR3 WT AS-NEPB2-PCR-2 reaction, except Actin-PCR products.

FIG. 5a. KrasP4 (G12V; G>T) NEPB or CBO blocker PCR on SW480 Cell line DNA: 8 out of 20 ul PCR products from the 20 ng DNA reaction were loaded on the gel. Clean PCR products were visualized on a 4% agarose gel from 0.1% mutant reactions and no PCR products were observed from WT reaction, except Actin-PCR products. The PCR product was also visualized from WT amplification without adding any Blockers. All of NTC was not undetermined.

FIG. 5b. KrasP7 (G13D; 13G>A) NEPB or CBO blocker PCR on HCT116 Cell line DNA: 8 ul PCR products were loaded on the gel. Clean PCR products were visualized from 0.1% mutant reactions and no PCR products were observed from WT reaction. The PCR product was visualized as well from WT amplification without adding any Blockers.

FIG. 6. EGFR Exon 21 L858R (2573 T>G) AS-NEPB-PCR on NCI-H1975 Cell line DNA: 8 ul PCR products were loaded on the gel. Clean PCR products were visualized from 0.1% mutant reactions and no PCR products were observed from WT reaction. The PCR product was visualized as well from WT amplification without adding any Blockers.

### DETAILED DESCRIPTION

With the aid of examples and exemplary discussions, disclosed herein is a novel edge-blocker oligonucleotide based Non-Extendable Primer Blocker Allele Specific-Real Time Polymerase Chain Reaction (“AS-NEPB-PCR”) based mutation assay methodology that overcomes the limitations of prior art approaches to enable amplification and detection of nucleic acid variants present at a frequency lower than 1% to achieve selectivity for targets present at levels of less than 1%. The disclosed method enables a universal design of Allele Specific primer and primer blocker that can be used in any of AS-RT-PCR assays to detect SNP or genetic mutations. The method simplifies assay optimization procedures and achieved 0.1% detection sensitivity with close to 100% specificity. The description starts with a detailed outline of experiments demonstrating the effectiveness of the technique.

### Materials and Methods

Cell Line and FFPE Tissue Samples

Cancer cell lines were ordered from American Type Culture Collection (ATCC, Manassas, Va. US) and cultured according ATCC protocols. The following cell lines containing specific allele sequences were used in this study: HT29 cell line (ATCC# HTB-38D) is heterozygous and SK-MEL28 cell line (ATCC# CRL-5908) is homozygous in BRAF mutation with predicted mutation effect of p.V600E (c.1799T>A). Characterization of BRAF mutation was described as likely oncogenic mutation (11). HCT 116 cell line (ATCC# CCL-247) has a mutation in codon 13 (p.G13D; c.G>A) and SW480 cell line (ATCC# CCL-228) has a mutation in codon 12 (p.G12V; c.G>T) of K-ras protooncogene. NCI-H1975 cell line (ATCC# CRL-5908) carries EGFR Exon 21 recurrent heterozygous missense mutation of L858R-2573T>G (12). SKBR3 cell line (ATCC# HTB-30) was used as wild type control for BRAF, K-ras and NCI-H358 cell line (ATCC# CRL-5807) as wild type control for EGFR mutation detection assays.

Melanoma and Colon tissue samples were purchased from ProteoGenex (Culver City, Calif. US), one of the providers of biological specimens.

Patient Samples

From 42 patients with metastatic colorectal cancer, 2×30 mL blood samples were taken for circulating tumor cells (CTC) enumeration and characterization by way of vena puncture before liver metastasis resection and prior to tumor manipulation. CTC enrichment and enumeration were processed by CellSearch system (Veridex LLC, Raritan, N.J.). All patients were included in the Erasmus Medical Center, Rotterdam, Netherlands after written informed consent was obtained.

DNA Extraction

Cell line DNA was extracted by using AllPrep™ DNA/RNA Micro Kit and FFPE tissue DNA was extracted by using RNeasy FFPE kit from Qiagen (Valencia Calif. US Cat#80284 and 74404) according to the manufacturer's instructions. Then, extracted DNA was quantified on Nanodrop-2000 Spectrophotometer (Thermo Fisher Scientific, Wilmington, Del. US) following the User Manual and stored at −20° C. until later use.

Oligo Design

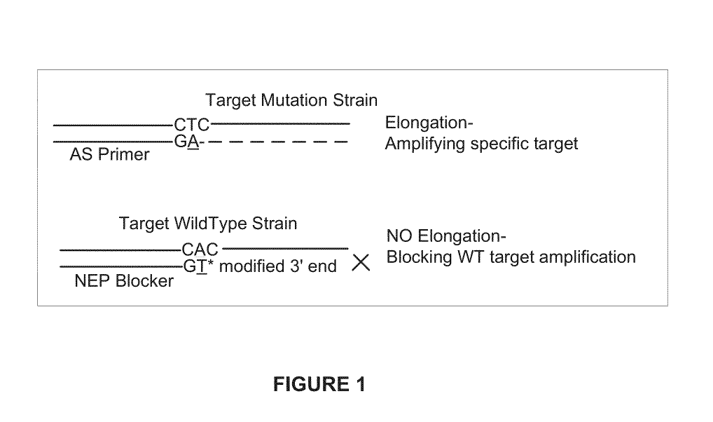

A general diagram for NEPB is demonstrated in FIG. 1. For an allele that was analyzed, allele-specific primers (ASP) can be designed to either positive or negative DNA (or RNA) strand. Either for the forward or the reverse primer, 3′-end is anchored on the variant base. The melting temperatures (Tm) of ASP should be close to the PCR extension temperature.

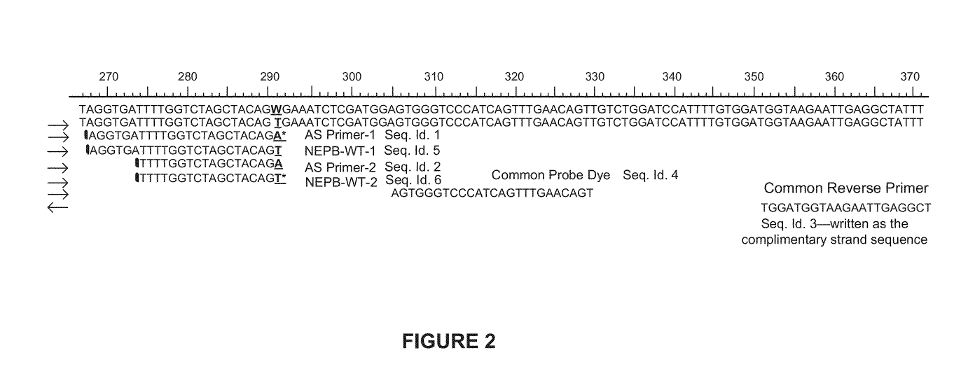

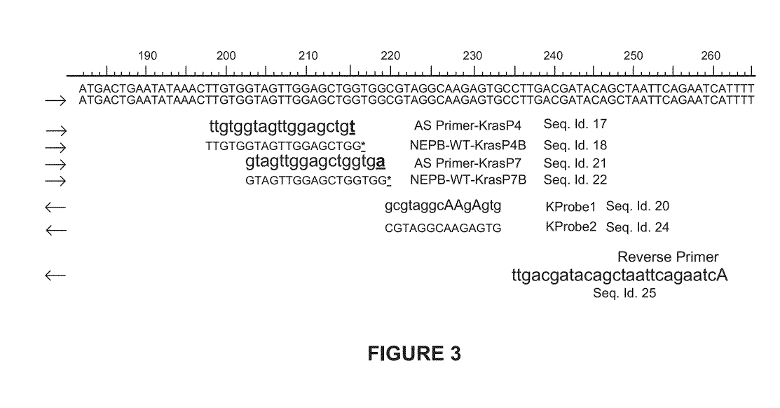

Edge-blocker oligonucleotide based AS-NEPB-PCR method was designed to the same strand and length as the allele-specific primer except the forward or reverse primer 3′-end is anchored on the WT base and not extendable by polymerases with 3′ end modification (phosphate or inverted dT or amino-C7). Center-blocker oligonucleotide design was based on the criteria listed in the paper (7) and used for comparison to edge-blocker oligonucleotide based AS-NEPB-PCR method. The design sequences were assembled by SeqMan II expert sequence analysis software (DNASTAR Inc, WI, US); FIG. 2 for the design of BRAF gene and FIG. 3 for two K-ras genes.

All of the designs including non-AS forward or reverse primers and probe used Oligo Primer Analysis Software from Molecular Biology Insights (Cascade, Colo. US). Tm was calculated by Oligo software based on PCR condition of 0.2 uM primers, 100 mM [Monovalent Cation] and 3 mM free Mg [2+].

All of the oligonucleotides, including primers, probe and blocker, were purchased from Biosearch Technologies, Inc (Novato, Calif. US), except MGB probes. Modified oligonucleotides including Fluorophore dye (FAM and CAL Fluor Orange) labeled at 5′ ends and BHQ or Phosphate at 3′ ends (Table 1a, b and C) were synthesized according to the manufacturer's instructions. Two MGB probes for K-ras assay were purchased from Applied Biosystems (Foster City, Calif.).

### AS-NEPB-PCR Amplification

The AS-NEPB-PCR assay for allele analysis of B-Raf, K-ras and EGFR included one ASP on the positive strand, one NEPB, one fluorescence labeled sequence specific TaqMan probe with BHQ or MGB at 3′ end and one non-AS reverse primer (RP) on the negative strand. The sequences of primers, probe and oligonucleotide blockers are listed in Table1a, Table1b and Table 1c below for the BRAF gene, Kras gene and EGFR gene respectively. All Tables are provided in the section titled ‘Tables’, which follows the ‘References’ section.

The assay was run as singlex or duplex AS-RT-PCR format, AS gene with Internal Control gene in two individual reactions or in one reaction, on Applied Biosystems 7500 (or 7900) Real-Time PCR System (Foster City, Calif.).

For BRAF p.V600E (c.1799T>A) detection, the final concentrations of each primer, blocker and probe of AS-NEPB-PCR assay are listed in Tables 2a. The assay was set up as follows: 10 ng to 50 ng of DNA heterogeneous mixture was used and was carried out in a final volume of 20 ul in reaction. The AS-NEPB-PCR was carried out using TaqMan® Gene Expression Master Kit (Applied Biosystems, Part #4368814). Each reaction consisted of 10.0 ul of 2×PCR Master Mix, 1 ul of 20× primer/blocker/probe mix, and 1-5 ul of 10 ng/ul total DNA sample. The AS-NEPB-PCR assays were run as follows: 1 cycle of denaturation at 95° C. for 10 min, 40 cycles of 95° C. for 20 seconds denaturation and 64° C. in favor of BRAF-NEPB1-PCR-1 or 58° C. in favor of BRAF-NEPB2-PCR-2 for 45 seconds annealing and extension under run Standard Mode.

To compare with the center-blocker oligonucleotide based AS-NEPB-PCR method, two BRAF center-blocker oligonucleotides (Table1a) designed based on the criteria listed in the publication (7) were tested. The final concentrations of center-blocker oligonucleotides were tested with 1×, 2× of the AS primer concentrations. The AS primer concentrations are 0.9 um with 0.9 um of reverse primers and 0.2 um of probe in a final 20 ul reaction. The PCR reagents and conditions are the same as the BRAF-NEPB1-PCR-1 assay. The concentrations of each primer, blocker and probe are listed in Table 2a.

For two K-ras mutations, the final concentrations of each primer, blocker oligonucleotides and probes of AS-NEPB-PCR assay are listed in Tables 1b and 2b. The assay was set up as follows: 20 ng of DNA heterogeneous mixture was used and was carried out in a final volume of 20 ul in reaction. The AS-NEPB-PCR was carried out using TaqMan® Gene Expression Master Kit. Each reaction consisted of 10.0 ul of 2×PCR Master Mix, 2 ul of 10× primer/blocker/probe mix, and 2 ul of 10 ng/ul total DNA sample. The AS-NEPB-PCR assays were run as follows: 1 cycle of denaturation at 95° C. for 10 min, 40 cycles of 95° C. for 20 seconds denaturation and 60° C. for 45 seconds annealing and extension under run Standard Mode.

The center-blocker oligonucleotide based AS-PCR method for K-ras was run at the same PCR condition as the edge-blocker oligonucleotide based AS-NEPB-PCR method except for using 4× center-blocker oligonucleotide concentration as corresponding ASP concentration, which was suggested in the publication (7).

For EGFR mutation, the final concentrations of each primer, blocker oligonucleotides and probes of AS-NEPB-PCR assay are listed in Tables 1c and 2c. The assay set-up was the same as K-ras mutation assay except DNA template. DNA samples were from NCI-H1975 and NCI-H358 heterogeneous mixture. The AS-NEPB-PCR assays were run as follows: 1 cycle of denaturation at 95° C. for 10 min, 40 cycles of 95° C. for 20 seconds denaturation and 63° C. for 45 seconds annealing and extension under run Standard Mode.

### Data Analysis

Edge-blocker oligonucleotide based AS-NEPB-PCR detection sensitivity/specificity of BRAF (V600E) and K-ras (G12V or G13D) were estimated by using dilutions of the related mutant cell line DNA (describe the above Cell Line Sample section) in wild-type DNA of the cell lines SKBR3. Dilutions were made at 5%, 1%, 0.5% and 0.1% mutant DNA and data were collected and analyzed by ABI 7500 fast System SDS software (Applied Biosystems). The same analysis method was used for both center-blocker oligonucleotide based AS-NEPB-PCR and edge-blocker oligonucleotide based AS-NEPB-PCR methods. Data was analyzed by manual threshold of 0.1 and baseline from 5 to 15 to obtain C_{T} value for both FAM and VIC channels. Assay was considered valid when Actin C_{T} value was less than or equal to 27, specific mutant gene C_{T} was less than or equal to 37 (˜3 copies) and all No Template Control (NTC) had undetectable C_{T}. PCR aliquots were also analyzed by agrose gel electrophoresis with 100 bases molecular marker (Invitrogen, Carlsbad, Calif.). One specific PCR product from a corresponding positive sample should be present after amplification.

### Sequence Analysis

Mutations detected by AS-NEPB-PCR in BRAF V600E were confirmed by direct sequencing using Rhodamine dye terminator cycle sequencing kit (Big Dye; Applied Biosystems). Cell line (20 ng) and FFPE (50 ng). DNA samples containing mutations were amplified by non-AS-PCR using sequence primers (Table1a) under the same PCR condition as AS-NEPB2-PCR-2. To verify the sequences, PCR amplified products were sent to GENEWIZ (South Plainfield, N.J., US). Sequencing was done on ABI 3730xl DNA Analyzer and analyzed using ABI PRISM DNA Sequencing Analysis Software (Applied Biosystems) according to the manufacturer's instructions.

### Results and Discussions

For BRAF V600E gene mutation detection, center-blocker Oligo (CBO) method was first adapted from the publication of K-ras mutation detection (7), the ASP and blocker designs were followed the criteria listed in the paper. Several assay conditions were tested in order to reach 0.1% detection sensitivity of BRAF mutation gene. We have tried to optimize assay conditions by titrated various annealing temperature (58, 60, 62, 64 and 65° C.) and ratio of ASP:PB (1:4, 1:2 and 1:1). However, none of conditions could reach 0.1% mutant detection sensitivity and without non-specific amplification on WT template. The results were observed under one of conditions for each CBO; 0.5% detection sensitivity was obtained without non-specific amplification from CBO-1 (64° C. and 1:1 ratio), however, the C_{T} in 0.5% has been shown great than 36. CBO-2 gave constantly non-specific amplification (64° C. and 1:2 ratio) if having 0.1% detection sensitivity (Table 3).

Under other conditions, AS-PCR reaction was blocked by increased concentration of the blocker or annealing temperature; and more non-specific amplification occurred when reducing the concentration of the blocker or annealing temperature (data not showed). In addition, CBO method required that the sequences of Primer, Blocker and Probe have to be partial-overlapping, blocker discriminating base in the middle of the oligonucleotide and different Tm (length), which bring about challenges for BRAF gene Oligo selection and assay condition optimization although the method was successful in the K-ras mutation assay.

Edge-blocker oligonucleotide (EBO) based AS-NEPB-PCR method was developed to improve detection sensitivity and remove non-specific amplification for BRAF gene mutation detection assay. Two sets of EBO, EBO-1 and EBO-2, with the corresponding forward AS primers, BRAF-AS-Forward Primer-1 and -2, were designed and evaluated with the BRAF V600E allelic variant. A common reverse primer and probe were designed downstream of the polymorphic site and used in AS-NEPB-PCR. A few of assay conditions were needed to be tested due to the same Tm for both ASP and NEPB; annealing temperature (64 and 65° C.) and ratio of ASP:EBO (1:1 or 1:2) for NE primer blocker-1 and annealing temperature (56, 58 and 60° C.) and ratio of ASP:EBO (7:1 or 3:1) for NE primer blocker-2. The annealing temperature screening was selected to be close to Tm of ASP (Table 1a). The ratio of ASP:EBO screening was decided based on the data generated from AS-PCR without adding up edge-blocker oligonucleotide (Table 3). BRAF-AS-Forward Primer-2 without edge-blocker oligonucleotide gave non-specific amplification when WT DNA was greater 50 ng input (data not shown), so less EBO was needed.

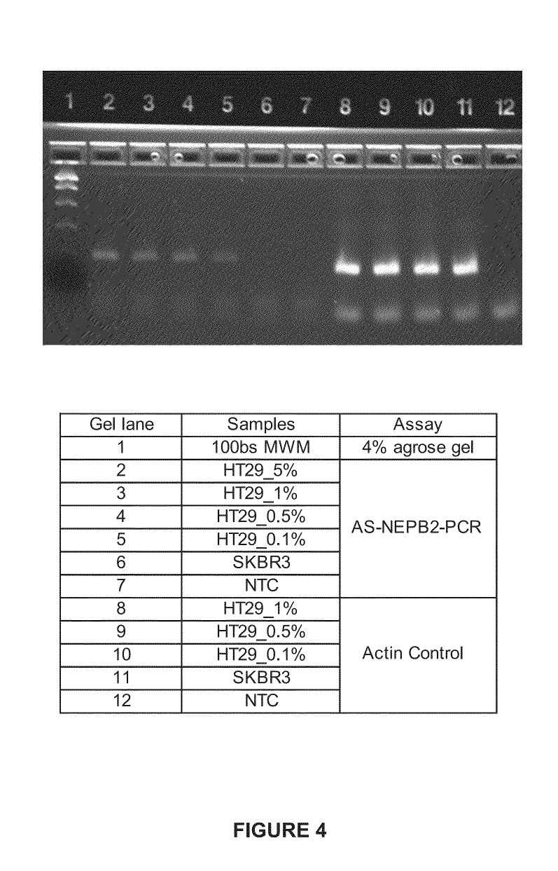

The results demonstrated that incorporation of edge-blocker oligonucleotide based AS-NEPB-PCR enhanced the sensitivity of the AS-PCR, without non-specific amplification on WT DNA. It performed better than CBO method (Table 3). Edge-blocker oligonucleotide based AS-NEPB-PCR method also showed strong allele specific amplifications, detected one copy of mutant DNA in 1000-copy normal DNA background of heterogeneous mixture (0.1% mutation frequency and 2-3 mutant copies) in both AS-NEPB-PCR assays. BRAF-AS-Forward Primer-2 with EBO-2 (AS-NEPB2-PCR-2) gave the best result to discriminate the wild type and mutant alleles, in which delta C_{T} is calculated as the difference between SKBR3 WT cell line C_{T} and the HT29 mutant/SKBR3 WT mixtures cell line C_{T}. Repeatable 0.1% mutant detection sensitivity (down to 3-5 copies of mutant) and undetectable WT specificity (up to 50 ng WT cell line DNA) were obtained by using AS-NEPB2-PCR-2 (Table 4). Undetectable WT specificity was also observed with 175 ng WT tissue DNA (data not shown). On gel image, single sharp bands were observed from 5% to 0.1% mutant reactions and no PCR products were observed from SKBR3 WT AS-NEPB2-PCR-2 reaction, except Actin-PCR products (FIG. 4).

Direct sequencing, as the gold standard, was used to verify the AS-NEPB2-PCR-2 method in both cell line and FFPE tissue DNA samples. The 100% sensitivity and specificity was obtained by using AS-NEPB-PCR method based on the sequence data (Table 5).

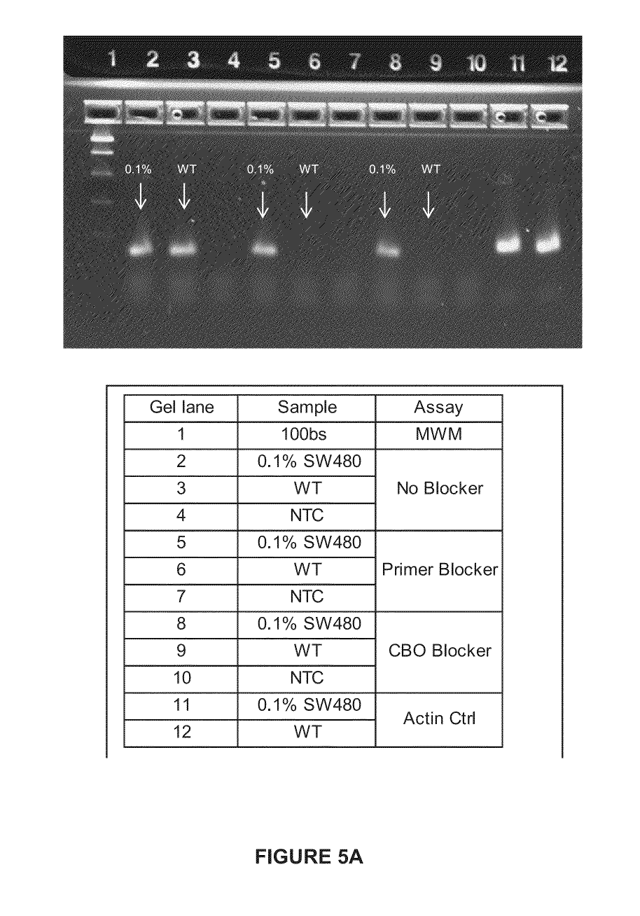

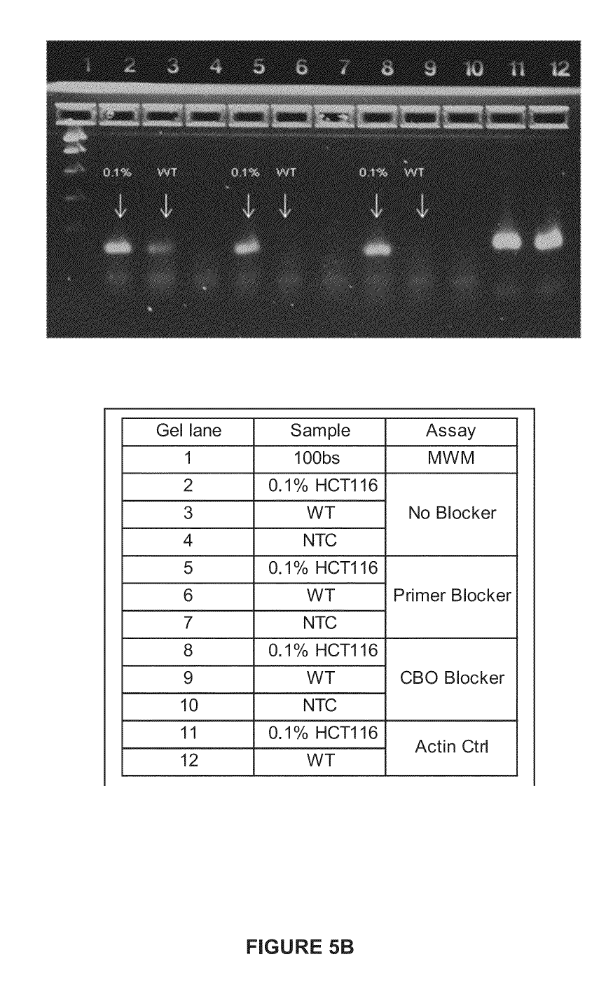

The edge-blocker oligonucleotide based AS-NEPB-PCR method was also verified on two KRAS gene mutants (p.G12V; G>T and p.G13D; 13G>A) and compared to the center-blocker oligonucleotide based AS-PCR method. A small number of ASP vs. edge-blocker oligonucleotide ratios were tested to obtain the best concentration of edge-blocker oligonucleotides. The same AS primers described in the paper were used under the annealing temperature 60° C. suggested by the paper (7). The best result was observed with 1:1 ratio of ASP to edge-blocker oligonucleotide for both Kras G12V and G13D mutant gene detection assays; 0.1% detection sensitivity (˜5 copies) without non-specific amplification on WT DNA (Tables 6a, 6b, and 6c). We have tested the edge-blocker oligonucleotide based AS-NEPB-PCR and center-blocker oligonucleotide based AS-PCR methods under the same reaction condition. Equivalent assay performances were obtained (Table 7 and FIGS. 5a and 5b). Good assay precision was obtained from the edge-blocker oligonucleotide based AS-NEPB-PCR method with <3% CV in three individual runs for two K-ras 0.1% (˜5 copies) mutants.

Edge-blocker oligonucleotides modified by inverted dT or amino-C7 were also evaluated. The equivalent assay performances were obtained as 3′ end modified by Phosphate (Table 8).

We have tested edge-blocker oligonucleotide based AS-NEPB-PCR method on 42 clinical samples, circulating colorectal tumor cells. BRAF (V600E) mutations were detected in two tissue samples and one CTC sample, which were matched with the sequencing data. Non-specific amplification was not observed in both tissue and CTC samples which confirmed by sequencing data (Table 9).

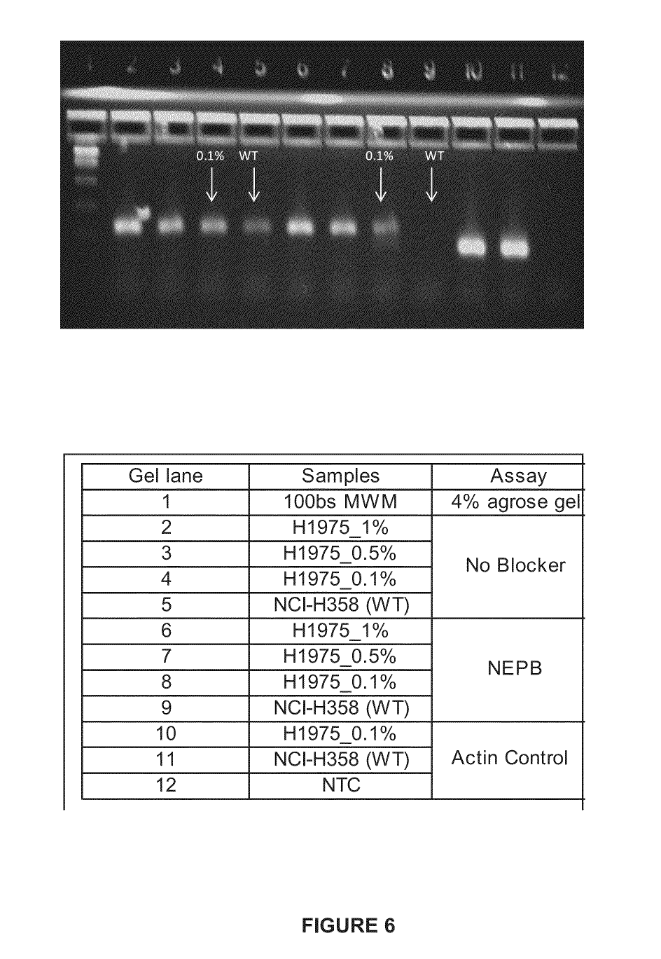

Edge-blocker oligonucleotide based AS-NEPB-PCR method was also evaluated on EGFR gene (exon 21_L858R) mutation detection. The results showed 0.1% of mutations (˜5 copies) were detected without non-specific amplification at 1:1 ratio of ASP to edge-blocker oligonucleotide and Annealing Temp 63° C. (Table 10 and FIG. 6). Good assay precision, <2% CV, was obtained from 5%, 1% and 0.1% in the triplicates.

Edge-blocker oligonucleotide based AS-NEPB-PCR method has been employed on the detection of 3 different genes (B-Raf, K-Ras, and EGFR) and 4 mutants (V600E, G12V, G13D and L858R) effectively. Optimal assay conditions were determined easily for each of the assays due to the advantage of edge-blocker oligonucleotide design, which has the same strand and length as the allele-specific primer producing almost the same melting temperatures (Tm) as ASP. We found that (1) normally 2 annealing temperatures are only needed beside Tm, one degree below and one degree up of ASP's Tm and (2) 1:1 ratio of ASP:blocker is suitable for most cases to get an optimal assay condition.

In conclusion, edge-blocker oligonucleotide based AS-NEPB-PCR method is a highly sensitive and specific method for mutation detection in highly heterogeneous samples. Also, the edge-blocker oligonucleotide based AS-NEPB-PCR method provides great advantages in simplifying assay design and assay optimization over the other blocking method. Edge-blocker oligonucleotide based AS-NEPB-PCR method allows an efficient workflow when a number of different mutation assays need to be developed.

### REFERENCES

1. Guttmacher, A. E. and F. S. Collins. 2002. Genomic medicine—a primer. N. Engl. J. Med. 347:1512-1520.
2. Phillips, K. A., D. L. Veenstra, E. Oren, J. K. Lee, and W. Sadee. 2001. Potential role of pharmacogenomics in reducing adverse drug reactions: a systematic review. JAMA 286: 2270-2279.
3. Newton, C. R., A. Graham, L. E. Heptinstall, S. J. Powell, C. Summers, N. Kalsheker, J. C. Smith, and A. F. Markham. 1989. Analysis of any point mutation in DNA. The amplification refractory mutation system (ARMS). Nucleic Acids Res. 17:2503-2516.
4. Livak, K. J., S. J. A. Flood, and J. A. Todd. 1995. Towards fully automated genome-wide polymorphism screening. Nat. Genet. 9:341-342.
5. Shale Dames and Karl V. Voelkerding at el, Characterization of Aberrant Melting Peaks in Unlabeled Probe Assays, Journal of Molecular Diagnostics, Vol. 9, No. 3, July 2007
6. Willem Maat and Pieter A. Van der Velden, Pyrophosphorolysis Detects B-RAF Mutations in Primary Uveal Melanoma, Investigative Ophthalmology & Visual Science, January 2008, Vol. 49, No. 1
7. Morlan J, Baker J, Sinicropi D, Mutation Detection by Real-Time PCR: A Simple, Robust and Highly Selective Method. PLoS ONE 4(2): e4584. doi:10.1371/journal.pone.0004584(2009)
8. Methods, Compositions, and Kits for Detecting Allelic Variants, Patent by life technologies corporation, International Application No.: PCT/US2010/028963
9. Susana Benlloch, et al. Detection of BRAF V600E Mutation in Colorectal Cancer. JMD November 2006, Vol. 8, No. 5
10. Tomoaki Tanaka at el, Frequency of and variables associated with the EGFR mutation and its subtypes. Int. J. Cancer: 126, 651-655 (2010) VC 2009
11. Ikediobi, O. N et al. Mutation Analysis of 24 Known Cancer Genes in the NCI-60 Cell Line Set Mol. Cancer Ther., 5(11):2606-2612, (2006)
12. Raffaella Sordella at el, Gefitinib-Sensitizing EGFR Mutations in Lung Cancer Activate Anti-Apoptotic Pathways. Science 305, 1163 (2004)

### TABLES

TABLE 1a
Primer/Probe/blocker Sequences and Labeling
for BRAF gene: for probes 5′ Modification
of FAM and CAL Fluor orange 560 and 3′
Modification of BHQ; blockers have 3′ end
phosphate modification.

| | BRAF | | | |
|---|---|---|---|---|
| Seq. | V600E | Sequence | Sequence | Tm |
| ID | Assay | Name | (5′-3″) | [° C.] |
| 1 | BRAF-AS- | BRAF- | AGGTGATTTTGG | 68.2 |
| | Forward | 268DF-AS | TCTAGCTACAGA | |
| | Primer-1 | | | |
| 2 | BRAF-AS- | BRAF- | TTTTGGTCTAGC | 58.1 |
| | Forward | 274DF-AS | TACAGA | |
| | Primer-2 | | | |
| 3 | Common | BRAF- | AGCCTCAATTCT | 71.5 |
| | Reverse | 347SR | TACCATCCA | |
| | Primer | | | |
| 4 | Common | BRAF- | FAM-AGTGGGTC | 79.0 |
| | Probe | 305DP | CCATCAGTTTGA | |
| | | | ACAGT-BHQ | |
| 5 | EBO NE | BRAF- | AGGTGATTTTGG | 68.2 |
| | Primer | 268DF-WTB | TCTAGCTACAG | |
| | blocker-1 | | T_PO₄ | |
| 6 | EBO NE | BRAF- | TTTTGGTCTAGC | 58.1 |
| | Primer | 274DF-WTB | TACAGT_PO₄ | |
| | blocker-2 | | | |
| 7 | CBO | BRAF- | TCTAGCTACAGT | 83.4 |
| | blocker-1 | 280ASB32 | GAAATCTCGATG | |
| | | | GAGTGGGT-PO₄ | |
| 8 | CBO | BRAF- | TCTAGCTACAGT | 68.3 |
| | blocker-2 | 280ASB23 | GAAATCTCGAT- | |
| | | | PO₄ | |
| 9 | Actin | B-actin | AAGCCACCCCAC | 71.6 |
| | (Internal | 3295U20 | TTCTCTCT | |
| 10 | Control) | B-actin | AATGCTATCACC | 71.4 |
| | | 3346L20 | TCCCCTGT | |
| 11 | | B-actin | Orange-AGAAT | 84.0 |
| | | 3319P26 | GGCCCAGTCCTC | |
| | | | TCCCAAGTC- | |
| | | | BHQ | |
| 12 | BRAF | BRAF- | TGATAGGAAAAT | 66.9 |
| | Outer | 186DF | GAGATCTACTGT | |
| 13 | Primer | BRAF- | TTTACATAAAAA | 67.0 |
| | (Nest- | 472DR | ATAAGAACACTG | |
| | PCR) | | ATT | |
| 14 | Sequence | BRAF- | GTGATTTTGGTC | 58.5 |
| | PCR | 270DF | TAGCTA | |
| 15 | Primer | BRAF- | AGCCTCAATTCT | 71.5 |
| | | 347SR | TACCATCCA | |
| 16 | Sequence | BRAF- | CTCAATTCTTAC | 69.6 |
| | Primer | 343SR | CATCCACAAA | |

TABLE 1b
Primer/Probe/blocker Sequences and Labeling
for K-ras gene: for probes 5′ Modification
of FAM and 3′ Modification of MGB; blockers
have 3′ end phosphate modification. Actin
primers and probe are the same as BRAF assay
in Table 1a. The melting temperatures (Tm)
were calculated by Oligo software as described
in “Oligo Design” for Tables 1a, 1b and 1c.

| | K-ras | | | |
|---|---|---|---|---|
| Seq | Mutation | Sequence | Sequence | Tm |
| ID | Assay | Name | (5′-3″) | [° C.] |
| 17 | G12V-AS | KrasP4 | TTGTGGTAGTTG | 66.3 |
| | Forward Primer | | GAGCTGT | |
| 18 | G12V-EBO NE | KrasP4B | TTGTGGTAGTTG | ~66.3 |
| | Primer blocker | | GAGCTGG-P04 | |
| 19 | G12V-CBO | ASB1 | TTGGAGCTGGTG | 77.9 |
| | blocker | | GCGTAGG-P04 | |
| 20 | G12V-Probe | KProbe1 | FAM-CACTCTTG | 65.9 |
| | | | CCTACGC-MGB | |
| 21 | G13D-AS Forward | KrasP7 | GTAGTTGGAGCT | 60.2 |
| | Primer | | GGTGA | |
| 22 | G13D-EBO NE | KrasP7B | GTAGTTGGAGCT | ~60.2 |
| | Primer blocker | | GGTGG-P04 | |
| 23 | G13D-CBO | ASB2 | GCTGGTGGCGTA | 74.8 |
| | blocker | | GGC-P04 | |
| 24 | G13D-Probe | KProbe2 | FAM-CACTCTTG | 59.9 |
| | | | CCTACG-MGB | |
| 25 | Common | KrasR | TGATTCTGAATT | 74.3 |
| | Reverse | | AGCTGTATCGTC | |
| | Primer | | AA | |

TABLE 1C
Primer/Probe/blocker Sequences and Labeling
for EGFR gene (L858R; 2573 T > G):
5′ Modification of FAM and 3′ Modification
of BHQ for probes; 3′ end phosphate
modification for blockers. Actin primer/probe
are the same as BRAF assay in Table 1a.

| | EGFR | | | |
|---|---|---|---|---|
| Seq | Mutation | Sequence | Sequence | Tm |
| ID | Assay | Name | (5′-3″) | (° C.) |
| 26 | Ex21_L858R | Ex21-248FAS | ATCACAGATTTT | 64.3 |
| | | | GGGCG | |
| 27 | | Ex21-248FWTB | ATCACAGATTTT | ~63.0 |
| | | | GGGCT-P04 | |
| 28 | | Ex21-330SR | GAAAATGCTGGC | 72.5 |
| | | | TGACCTAAA | |
| 29 | | Ex21-271P | FAM-TGGGTGCG | 79.6 |
| | | | GAAGAGAAAGAA | |
| | | | TACC-BHQ | |

TABLE 2a
Concentration of primer/blocker/probe in
AS-NEPB-PCR BRAF V600E duplex assay

| | | Final |
|---|---|---|
| BRAF V600E NEPB | | Conc. |
| Assay | Name(Primer/Blocker/Probe) | (uM) |
| BRAF-AS-NEPB1-PCR-1 | BRAF-268DF-AS (SEQ ID 1) | 0.450 |
| | BRAF-347SR (SEQ ID 3) | 0.450 |
| | BRAF-268DF-WTB (SEQ ID 5) | 0.900 |
| | BRAF-305DP (SEQ ID 4) | 0.250 |
| BRAF-AS-NEPB2-PCR-2 | BRAF-274DF-AS (SEQ ID 2) | 0.900 |
| | BRAF-347SR (SEQ ID 15) | 0.900 |
| | BRAF-274DF-WTB (SEQ ID 6) | 0.125 |
| | BRAF-305DP (SEQ ID 4) | 0.250 |
| Actin | B-actin 3295U20 (SEQ ID 9) | 0.030 |
| | B-actin 3346L20 (SEQ ID 10) | 0.030 |
| | B-actin 3319P26_Ora (SEQ ID | 0.030 |
| | 11) | |

TABLE 2b
Concentration of primer/blocker/probe in AS-NEPB-PCR K-ras singlex
assay

| | | Final |
|---|---|---|
| | | Conc. |
| K-ras Mutation Assay | Name(Primer/Blocker/Probe) | (uM) |
| G12V-NE Primer Blocker | KrasP4 (SEQ ID 17) | 0.90 |
| | KrasR (SEQ ID 25) | 0.90 |
| | KrasP4B (SEQ ID 18) | 0.90 |
| | KProbe1 (SEQ ID 20) | 0.20 |
| G13D-NE Primer Blocker | KrasP7 (SEQ ID 21) | 0.90 |
| | KrasR (SEQ ID 25) | 0.90 |
| | KrasP7B (SEQ ID 22) | 0.90 |
| | KProbe2 (SEQ ID 24) | 0.20 |
| Actin | B-actin 3295U20 (SEQ ID 9) | 0.40 |
| | B-actin 3346L20 (SEQ ID 10) | 0.40 |
| | B-actin 3319P26_Ora (SEQ ID 11) | 0.20 |

TABLE 2C
Concentration of primer/blocker/probe in
AS-NEPB-PCR EGFR singlex assay

| EGFR | | |
|---|---|---|
| Mutation Assay | Name(Primer/Blocker/Probe) | Final Conc. (uM) |
| Ex21_L858R | Ex21_248FAS (SEQ ID 26) | 0.90 |
| | Ex21-248FWTB (SEQ ID 27) | 0.90 |
| | Ex21-330SR (SEQ ID 28) | 0.90 |
| | Ex21-271P (SEQ ID 29) | 0.20 |
| Actin | B-actin 3295U20 (SEQ ID 9) | 0.40 |
| | B-actin 3346L20 (SEQ ID 10) | 0.40 |
| | B-actin 3319P26_Ora (SEQ ID 11) | 0.20 |

TABLE 3
10 ng DNA of HT29 mutant/SKBR3 WT mixtures at various ratios
were input into the PCR reactions for BRAF V600E mutant
detection, 0.1% of mixtures equivalent to ~3 copies of mutant.
Undetermined in PCR was considered as C_{T} 40 during delta
C_{T} calculations. Both NE Primer blocker, EBO, method
reached 0.1% detection sensitivity without non-specific amplification.
NE Primer blocker-2 showed the best results to discriminate alleles
(bigger delta C_{T} between WT and mutant) compared to
CBO blocker method. CBO blocker-1 showed 0.5% detection
sensitivity and non-specific amplification on WT DNA. However, the
delta C_{T} between WT and mutant was ~3Ct less than NEPB
method. CBO blocker-2 gave constantly non-specific amplification if
having 0.1% detection sensitivity. All the final reaction conditions
were described in the Method of “AS-NEPB-PCR Amplification”.

| | No | NE Primer | NE Primer | CBO | CBO |
|---|---|---|---|---|---|
| | blocker | blocker-1 | blocker-2 | blocker-1 | blocker-2 |
| % Mutant/ | | | | | |
| PCR | | | | | |
| Result | | | | | |
| 5% | 28.9 | 31.2 | 32.6 | 33.5 | 34.2 |
| 1% | 28.7 | 31.1 | 32.3 | 34.6 | 39.9 |
| 0.5% | 29.0 | 30.9 | 32.1 | 34.6 | Undetermined |
| 0.1% | 29.3 | 31.3 | 33.3 | 34.4 | Undetermined |
| Mutant | | | | | |
| Delta | | | | | |
| C_{T} to WT | | | | | |
| 5% | 2.9 | 7.4 | 8.5 | 5.5 | 3.3 |
| 1% | 1.6 | 3.8 | 5.9 | 3.6 | 3.3 |
| 0.5% | 0.7 | 3.6 | 6.1 | 2.6 | 2.2 |
| 0.1% | 0.5 | 2.2 | 3.1 | −1.0 | 1.2 |

TABLE 4
10-50 ng DNA of HT29 mutant/SKBR3 WT mixtures at various ratios
were input into AS-NEPB2-PCR-2 of BRAF V600E mutant detection.
The method detected constantly 0.1% of mutation rate without
non-specific amplification. All of Actin C_{T}s were <25 and
NTC was not undetermined.

| % Mutant/PCR | | | |
|---|---|---|---|
| Result (C_{T}) | 10 ng | 20 ng | 50 ng |
| 5.0% | 31.5 | 28.5 | 27.5 |
| 1.0% | 34.1 | 31.9 | 30.2 |
| 0.5% | 33.9 | 32.1 | 31.7 |
| 0.1% | 36.9 | 33.6 | 33.6 |
| WT | Undetermined | Undetermined | Undetermined |

TABLE 5
The assay gave consistent performance on detecting the mutated
allele without non-specific amplification of DNA samples, which
were confirmed by sequencing. NVD-No variants detected.

| | | | Sequence Data |
|---|---|---|---|
| | | | (p.V600E |
| | BRAF (C_{T}) | ACTIN (C_{T}) | GTG > GAG) |
| Melanoma FFPE | | | |
| 13820T2 | 26.6 | 24.5 | 1. T > A(50%) |
| 13819T2 | 25.5 | 24.1 | 2. T > A(50%) |
| 13788T2 | 28.5 | 24.9 | 3. T > A(50%) |
| 13724T2 | 38.6 | 26.6 | 4. p.V600K |
| | | | c.1798_1799 |
| | | | GT > AA |
| | | | (Complex60%) |
| Colon FFPE | | | |
| 02671T1 | Undetermined | 22.7 | 5. NVD |
| 02671T2 | Undetermined | 24.1 | 6. NVD |
| 02973T1 | Undetermined | 24.8 | 7. NVD |
| 02973T2 | Undetermined | 25.7 | 8. NVD |
| Cell Line DNA | | | |
| H29_40% | 26.7 | 24.4 | 9. T > A(40%) |
| H29_20% | N/A | N/A | 10. T > A(20%) |
| H29_10% | 27.2 | 23.2 | 11. T > A(10%) |
| WT_SKBR3 | Undetermined | 24.9 | 12. NVD |

TABLE 6a
KrasP4 (G12V; G > T) AS-NEPB-PCR on SW480 Cell line
DNA: 20 ng DNA of SW480 mutant/SKBR3 WT mixtures at three
ASP:NEPB ratios were input into the PCR reactions, 0.1% of
mixtures equivalent to ~5 copies of mutant. PCR conditions
were as stated previously. The ratio of 1:1 gave the best result that
reached 0.1% detection sensitivity without non-specific
amplification. All of NTC was not undetermined.

| NEPB | | | | | |
|---|---|---|---|---|---|
| Titration/ | | | | | |
| AS-PCR (C_{T}) | 5% | 1% | 0.5% | 0.1% | WT |
| No Blocker | 28.9 | 31.2 | 32.6 | 33.5 | 34.2 |
| 2:1-ASP:NEPB | 28.7 | 31.1 | 32.3 | 34.6 | 39.9 |
| 1:1-ASP:NEPB | 29.0 | 30.9 | 32.1 | 34.6 | Undetermined |
| 1:2-ASP:NEPB | 29.3 | 31.3 | 33.3 | 34.4 | Undetermined |
| Actin (Ctrl) | 22.8 | 22.8 | 22.5 | 23.1 | 22.8 |

TABLE 6b
KrasP7 (G13D; 13G > A) AS-NEPB-PCR on HCT116 Cell line
DNA: 20 ng DNA of HCT116 mutant/SKBR3 WT mixtures with three
ASP:NEPB ratios were tested. PCR condition was the same as
KrasP4 assay. The ratio of 2:1 or 1:1 reached 0.1% detection
sensitivity without non-specific amplification (ratio of 1:1 showed
the similar C_{T} value as ratio of 2:1, 1:1 ratio was selected
for the further study). All of NTC was not undetermined.

| NEPB | | | | | |
|---|---|---|---|---|---|
| Titration/ | | | | | |
| AS-PCR (C_{T}) | 5% | 1% | 0.5% | 0.1% | WT |
| No Blocker | 30.6 | 34.1 | 33.8 | 35.0 | 38.7 |
| 2:1-ASP:NEPB | 31.4 | 32.9 | 35.4 | 37.6 | Undetermined |
| 1:1-ASP:NEPB | 31.6 | 33.9 | 34.0 | 36.5 | Undetermined |
| 1:2-ASP:NEPB | 32.2 | 33.4 | 33.8 | 38.2 | Undetermined |
| Actin (Ctrl) | 22.4 | 22.8 | 22.7 | 22.6 | 22.9 |

TABLE 7
NEPB and CBO Blocker methods were tested on SW480 and HCT116 Cell
line DNA with ASP of KrasP4 and KrasP7: 20 ng DNA of each mutant/SKBR3 WT
mixtures with 1:1 ratio of ASP:NEPB or 1:4 ratio of ASP:CBO blocker was used
the PCR reactions. PCR conditions were as stated previously. NEPB method
showed the equivalent, if not better, assay performances as CBO Blocker
method; reached 0.1% detection sensitivity without non-specific amplification on
WT DNA. All of NTC was not undetermined.

| K-rasP4 (G12V; G > T) | 5% | 1% | 0.5% | 0.1% | |
|---|---|---|---|---|---|
| AS-PCR (C_{T}) | SW480 | SW480 | SW480 | SW480 | WT |
| No Blocker | 28.9 | 31.2 | 32.6 | 33.0 | 34.2 |
| 1:1-ASP:NEPB | 28.6 | 30.8 | 32.7 | 34.2 | Undetermined |
| 1:4-ASP:CBO | 29.3 | 31.3 | 33.3 | 22.5 | Undetermined |
| blocker | | | | | |
| Actin (Ctrl) | 23.1 | 22.7 | 22.7 | 22.8 | 22.9 |
| K-rasP7 (G13V; | | | | | |
| 13G > A) | 5% | 1% | 0.5% | 0.1% | |
| AS-PCR (C_{T}) | HCT116 | HCT116 | HCT116 | HCT116 | WT (SKBR3) |
| No Blocker | 30.6 | 34.1 | 33.8 | 35.0 | 38.7 |
| 1:1-ASP:NEPB | 30.6 | 33.2 | 35.0 | 36.1 | Undetermined |
| 1:4-ASP:CBO | 30.6 | 33.2 | 34.1 | 37.2 | Undetermined |
| blocker | | | | | |
| Actin (Ctrl) | 23.1 | 22.7 | 22.7 | 22.8 | 22.9 |

TABLE 8
20 ng DNA of HT29 mutant (BRAF V600E)/SKBR3 WT mixtures
at various ratios were input into AS-NEPB-PCR. Three NEPB
modifications, Phosphate or inverted dT or amino-C7, gave
equivalent results. The method detected 0.1% of mutation rate
without non-specific amplification. All of Actin Cts were <25
and NTC was not undetermined.

| % | | | | |
|---|---|---|---|---|
| Mutant/PCR | No | | | |
| Result (C_{T}) | Blocker | Phosphate | Inverted dT | Amino-C7 |
| 5% | 28.9 | 29.2 | 28.7 | 29.0 |
| 1% | 31.7 | 31.4 | 30.7 | 32.1 |
| 0.5% | 32.6 | 33.9 | 33.2 | 33.3 |
| 0.1% | 35.6 | 35.7 | 36.7 | 36.8 |
| WT | 36.1 | Undetermined | Undetermined | Undetermined |
| (SKBR3) | | | | |

TABLE 9
Using AS-NEPB-PCR method, BRAF (V600E) mutations were
detected in the two clinical tissue samples (2/42) and one CTC
sample (1/42), which were 100% (tissues) or 50% (CTCs),
matched with sequencing data. For CTC samples, the mutation
only was detected for CTC-196 which had 5 CTCs, but not
detected for CTC-220 which had no CTC account based on the
analysis of CellSearch system. Non-specific amplification was
not observed in both tissue and CTC samples which were
confirmed by their sequence data. All of Actin C_{T}s
were <25 (data not shown).

| Sample | Matched | Tissue | Tissue | Tissue PCR | CTC PCR |
|---|---|---|---|---|---|
| # | CTC ID | ID | Sequence Data | data (Ct) | data (Ct) |
| 1 | CTC207 | 8090 | ND | ND | ND |
| 2 | CTC249 | 8110 | ND | ND | ND |
| 3 | CTC253 | 5439 | ND | ND | ND |
| 4 | CTC209 | 8086 | ND | ND | ND |
| 5 | CTC252 | 5426 | ND | ND | ND |
| 6 | CTC195 | 5419 | ND | ND | ND |
| 7 | CTC202 | 8085 | ND | ND | ND |
| 8 | CTC194 | 5423 | ND | ND | ND |
| 9 | CTC192 | 8109 | ND | ND | ND |
| 10 | CTC218 | 8094 | ND | ND | ND |
| 11 | CTC211 | 8101 | ND | ND | ND |
| 12 | CTC220 | 8095 | c.T > A; | 26.3 | ND |
| | | | p.V600E, 10% | | |
| 13 | CTC247 | 8103 | ND | ND | ND |
| 14 | CTC191 | 5416 | ND | ND | ND |
| 15 | CTC216 | 8093 | ND | ND | ND |
| 16 | CTC198 | 8082 | ND | ND | ND |
| 17 | CTC246 | 8106 | ND | ND | ND |
| 18 | CTC201 | 8084 | ND | ND | ND |
| 19 | CTC189 | 8108 | ND | ND | ND |
| 20 | CTC190 | 5454 | ND | ND | ND |
| 21 | CTC215 | 8102 | ND | ND | ND |
| 22 | CTC227 | 8100 | ND | ND | ND |
| 23 | CTC243 | 5445 | ND | ND | ND |
| 24 | CTC196 | 5414 | c.T > A; | 30.3 | 34.6 |
| | | | p.V600E, 80% | | |
| 25 | CTC200 | 8083 | ND | ND | N/A |
| 26 | CTC254 | 8118 | ND | ND | ND |
| 27 | CTC221 | 8097 | ND | ND | ND |
| 28 | CTC204 | 8089 | ND | ND | ND |
| 29 | CTC219 | 8096 | ND | ND | ND |
| 30 | CTC250 | 8112 | ND | ND | ND |
| 31 | CTC251 | 8114 | ND | ND | ND |
| 32 | CTC244 | 5512 | ND | ND | ND |
| 33 | CTC210 | 8087 | ND | ND | ND |
| 34 | CTC248 | 8107 | ND | ND | ND |
| 35 | CTC222 | 8098 | ND | ND | ND |
| 36 | CTC226 | 5438 | ND | ND | ND |
| 37 | CTC217 | 5431 | Seq Fail | ND | ND |
| 38 | CTC245 | 8105 | ND | 39.5 | ND |
| 39 | CTC208 | 8091 | ND | ND | N/A |
| 40 | CTC284 | 8116 | ND | ND | ND |
| 41 | CTC203 | 8078 | ND | ND | ND |
| 42 | CTC225 | 8099 | ND | 39.4 | ND |

TABLE 10
EGFR Exon 21 L858R (2573 T > G) AS-NEPB-PCR on NCI-H1975
Cell line DNA: 20 ng DNA of NCI-H1975 mutant/NCI-H358 DNA WT
mixtures at 1:1 ASP:NEPB ratios were input into the PCR reactions,
0.1% of mixtures equivalent to ~5 copies of mutant. PCR conditions
were as stated previously. 0.1% detection sensitivity was reached
without non-specific amplification. All of NTC was not undetermined.

| % Mutant/PCR | | | | | |
|---|---|---|---|---|---|
| Result (C_{T}) | 5% | 1% | 0.5% | 0.1% | WT |
| No Blocker | 29.2 | 29.5 | 31.0 | 33.0 | 35.5 |
| AS-NEPB-PCR | 28.3 | 29.8 | 31.3 | 34.2 | Undetermined |
| Actin (Ctrl) | 22.4 | 22.9 | 22.4 | 22.5 | 22.3 |

I claim:

1. A method for detecting, in the presence of a wild type sequence, a mutant nucleic acid sequence defined by one or more mutation due to at least one or more of a substitution, a deletion or an insertion at a position while suppressing the signal due to the wild type sequence, the method comprising the steps of:
selecting an allele specific primer corresponding to a portion of the mutant nucleic acid sequence such that a 3′ end of the allele specific primer aligns with at least one mutated nucleic acid position without a mismatch;
selecting an edge-blocker wild type oligonucleotide corresponding to the wild type sequence such that the 3′ end of the edge-blocker wild type oligonucleotide has at least one mismatch at or about its 3′ end, and, wherein, furthermore maybe similar as CBO method, the 3′ end of the edge-blocker wild type oligonucleotide is blocked whereby making it non-extendable in a polymerase chain reaction;
selecting one or more reverse primers;
selecting one or more probes to detect amplification products of the polymerase chain reaction;
carrying out the polymerase chain reaction with the ingredients comprising the allele specific primer, the edge-blocker wild type oligonucleotide, the one or more reverse primers and the one or more probes.

2. The method of claim 1 wherein at least one of the one or more probes is added after the polymerase chain reaction is initiated.

3. The method of claim 1 wherein the mutant nucleic acid sequence and the wild type sequence are generated with a reverse transcriptase.

4. The method of claim 1 wherein the one or more mutation includes two adjacent point mutations.

5. The method of claim 1 wherein the edge-blocker wild type oligonucleotide has a mismatch in at least one of the three base pairs immediately adjacent to its blocked 3′ end.

6. The method of claim 1 wherein the edge-blocker wild type oligonucleotide is equal in length to the allele specific primer.

7. The method of claim 1 wherein the edge-blocker wild type oligonucleotide is longer than the allele specific primer at its 5′ end.

8. The method of claim 8 wherein the melting temperature of the allele specific primer is—equal or higher than that for the edge-blocker wild type oligonucleotide relative to the wild-type sequence.

9. The method of claim 8 wherein the melting temperature of the allele specific primer is lower than that for the edge-blocker wild type oligonucleotide relative to the wild-type sequence.

10. The method of claim 1 wherein the edge-blocker wild type oligonucleotide is present at an equal or lower concentration than the allele specific primer whereby blocking the amplification of the wild type sequence during the polymerase chain reaction.

11. The method of claim 1 wherein the detection of the mutant nucleic acid sequence includes quantitation to estimate a level of the mutant nucleic acid sequence relative to the wild type sequence.

12. The method of claim 1 wherein the mutant nucleic acid sequence corresponds to a tumor cell type.

13. The method of claim 1 wherein the mutant nucleic acid sequence corresponds to a metastatic cell disease.

14. A diagnostic method for early detection of cancer by way to detecting the presence of one or more mutant cells, in a sample derived from a patient, harboring a mutant nucleic acid sequence in the presence of a wild type cells, the method comprising the steps of:
selecting an allele specific primer corresponding to a portion of the mutant nucleic acid sequence such that the 3′ end of the allele specific primer does not have a mismatch while being aligned with at least one mutated nucleic acid position;
selecting a edge-blocker wild type oligonucleotide corresponding to the wild type sequence such that the 3′ end of the edge-blocker wild type oligonucleotide has at least one mismatch at or about its 3′ end, and, wherein, furthermore, the 3′ end of the edge-blocker wild type oligonucleotide is blocked whereby making it non-extendable in a polymerase chain reaction;
selecting one or more reverse primers;
selecting one or more probes to detect amplification products of the polymerase chain reaction;
carrying out the polymerase chain reaction with the ingredients comprising the allele specific primer, the edge-blocker wild type oligonucleotide, the one or more reverse primers and the one or more probes; and
detecting an early stage of cancer if the mutant nucleic acid sequence is present in the sample.

15. The method of claim 15 wherein the mutant nucleic acid sequence's presence is detected if amplification products corresponding to it are detected in less than a pre-specified number of amplification cycles.

16. The method of claim 15 wherein the mutant nucleic acid sequence's presence is detected if amplification products corresponding to it are detected and a reference mutant sequence is not detected in the same sample.

17. The method of claim 1 or 15 wherein the 3′ end of the edge-blocker wild type oligonucleotide is blocked from extension in a PCR reaction because it does not have a hydroxyl group.

18. The method of claim 18 wherein the 3′ end of the edge-blocker wild type oligonucleotide is blocked by derivatizing or replacing its 3′ hydroxyl group with one or more selected from the group consisting of phosphate, inverted dT and amino-C7.


SEQUENCE LISTING (36 sequences)

SEQ ID NO: 1 - DNA, 24 bp, Artificial Sequence
  Description of Artificial Sequence Synthetic oligonucleotide
aggtgattttggtctagctacaga

SEQ ID NO: 2 - DNA, 18 bp, Artificial Sequence
  Description of Artificial Sequence Synthetic oligonucleotide
ttttggtctagctacaga

SEQ ID NO: 3 - DNA, 21 bp, Artificial Sequence
  Description of Artificial Sequence Synthetic oligonucleotide
agcctcaattcttaccatcca

SEQ ID NO: 4 - DNA, 25 bp, Artificial Sequence
  Description of Artificial Sequence Synthetic oligonucleotide
agtgggtcccatcagtttgaacagt

SEQ ID NO: 5 - DNA, 24 bp, Artificial Sequence
  Description of Artificial Sequence Synthetic oligonucleotide
aggtgattttggtctagctacagt

SEQ ID NO: 6 - DNA, 18 bp, Artificial Sequence
  Description of Artificial Sequence Synthetic oligonucleotide
ttttggtctagctacagt

SEQ ID NO: 7 - DNA, 32 bp, Artificial Sequence
  Description of Artificial Sequence Synthetic oligonucleotide
tctagctacagtgaaatctcgatggagtgggt

SEQ ID NO: 8 - DNA, 23 bp, Artificial Sequence
  Description of Artificial Sequence Synthetic oligonucleotide
tctagctacagtgaaatctcgat

SEQ ID NO: 9 - DNA, 20 bp, Artificial Sequence
  Description of Artificial Sequence Synthetic oligonucleotide
aagccaccccacttctctct

SEQ ID NO: 10 - DNA, 20 bp, Artificial Sequence
  Description of Artificial Sequence Synthetic oligonucleotide
aatgctatcacctcccctgt

SEQ ID NO: 11 - DNA, 26 bp, Artificial Sequence
  Description of Artificial Sequence Synthetic oligonucleotide
agaatggcccagtcctctcccaagtc

SEQ ID NO: 12 - DNA, 24 bp, Artificial Sequence
  Description of Artificial Sequence Synthetic oligonucleotide
tgataggaaaatgagatctactgt

SEQ ID NO: 13 - DNA, 27 bp, Artificial Sequence
  Description of Artificial Sequence Synthetic oligonucleotide
tttacataaaaaataagaacactgatt

SEQ ID NO: 14 - DNA, 18 bp, Artificial Sequence
  Description of Artificial Sequence Synthetic oligonucleotide
gtgattttggtctagcta

SEQ ID NO: 15 - DNA, 21 bp, Artificial Sequence
  Description of Artificial Sequence Synthetic oligonucleotide
agcctcaattcttaccatcca

SEQ ID NO: 16 - DNA, 22 bp, Artificial Sequence
  Description of Artificial Sequence Synthetic oligonucleotide
ctcaattcttaccatccacaaa

SEQ ID NO: 17 - DNA, 19 bp, Artificial Sequence
  Description of Artificial Sequence Synthetic oligonucleotide
ttgtggtagttggagctgt

SEQ ID NO: 18 - DNA, 19 bp, Artificial Sequence
  Description of Artificial Sequence Synthetic oligonucleotide
ttgtggtagttggagctgg

SEQ ID NO: 19 - DNA, 19 bp, Artificial Sequence
  Description of Artificial Sequence Synthetic oligonucleotide
ttggagctggtggcgtagg

SEQ ID NO: 20 - DNA, 15 bp, Artificial Sequence
  Description of Artificial Sequence Synthetic oligonucleotide
cactcttgcctacgc

SEQ ID NO: 21 - DNA, 17 bp, Artificial Sequence
  Description of Artificial Sequence Synthetic oligonucleotide
gtagttggagctggtga

SEQ ID NO: 22 - DNA, 17 bp, Artificial Sequence
  Description of Artificial Sequence Synthetic oligonucleotide
gtagttggagctggtgg

SEQ ID NO: 23 - DNA, 15 bp, Artificial Sequence
  Description of Artificial Sequence Synthetic oligonucleotide
gctggtggcgtaggc

SEQ ID NO: 24 - DNA, 14 bp, Artificial Sequence
  Description of Artificial Sequence Synthetic oligonucleotide
cactcttgcctacg

SEQ ID NO: 25 - DNA, 26 bp, Artificial Sequence
  Description of Artificial Sequence Synthetic oligonucleotide
tgattctgaattagctgtatcgtcaa

SEQ ID NO: 26 - DNA, 17 bp, Artificial Sequence
  Description of Artificial Sequence Synthetic oligonucleotide
atcacagattttgggcg

SEQ ID NO: 27 - DNA, 17 bp, Artificial Sequence
  Description of Artificial Sequence Synthetic oligonucleotide
atcacagattttgggct

SEQ ID NO: 28 - DNA, 21 bp, Artificial Sequence
  Description of Artificial Sequence Synthetic oligonucleotide
gaaaatgctggctgacctaaa

SEQ ID NO: 29 - DNA, 24 bp, Artificial Sequence
  Description of Artificial Sequence Synthetic oligonucleotide
tgggtgcggaagagaaagaatacc

SEQ ID NO: 30 - DNA, 105 bp, Artificial Sequence
  Description of Artificial Sequence Synthetic polynucleotide
taggtgattttggtctagctacagwgaaatctcgatggagtgggtcccatcagtttgaacagttgtctggatccattttgtggatggtaagaattgaggctattt

SEQ ID NO: 31 - DNA, 105 bp, Artificial Sequence
  Description of Artificial Sequence Synthetic polynucleotide
taggtgattttggtctagctacagtgaaatctcgatggagtgggtcccatcagtttgaacagttgtctggatccattttgtggatggtaagaattgaggctattt

SEQ ID NO: 32 - DNA, 21 bp, Artificial Sequence
  Description of Artificial Sequence Synthetic primer
tggatggtaagaattgaggct

SEQ ID NO: 33 - DNA, 84 bp, Artificial Sequence
  Description of Artificial Sequence Synthetic
atgactgaatataaacttgtggtagttggagctggtggcgtaggcaagagtgccttgacgatacagctaattcagaatcatttt

SEQ ID NO: 34 - DNA, 15 bp, Artificial Sequence
  Description of Artificial Sequence Synthetic probe
gcgtaggcaagagtg

SEQ ID NO: 35 - DNA, 14 bp, Artificial Sequence
  Description of Artificial Sequence Synthetic probe
cgtaggcaagagtg

SEQ ID NO: 36 - DNA, 26 bp, Artificial Sequence
  Description of Artificial Sequence Synthetic primer
ttgacgatacagctaattcagaatca